(19) United States
(12) Patent Application Publication
(10) Pub. No.: US 2025/0262260 A1
(43) Pub. Date: Aug. 21, 2025

(54) USE OF TURNIP EXTRACT IN THE PREPARATION OF A DRUG FOR THE PREVENTION AND TREATMENT OF ALZHEIMER'S DISEASE

(71) Applicant: Xinjiang University, Xinjiang (CN)

(72) Inventors: Jing MIAO, Xinjiang (CN); Jinyao LI, Xinjiang (CN); Jin WANG, Xinjiang (CN)

(21) Appl. No.: 19/027,376

(22) Filed: Jan. 17, 2025

(30) Foreign Application Priority Data
Feb. 20, 2024 (CN) 202410191238.0

(51) Int. Cl. A61K 36/31; A61P 25/28; B01D 11/02; B01D 11/04

(52) U.S. Cl. CPC A61K 36/31; A61P 25/28; B01D 11/0265; B01D 11/0288; B01D 11/0492; A61K 2236/333; A61K 2236/35; A61K 2236/51

(57) ABSTRACT

The present disclosure provides use of a turnip extract in the preparation of a drug for the prevention and treatment of Alzheimer's disease, which belongs to the technical field of biomedicine. The turnip extract of the present disclosure has good activity against Alzheimer's disease and has good application prospects in a drug for the prevention and treatment of Alzheimer's disease.

### CROSS-REFERENCE TO RELATED APPLICATION

This patent application claims priority to Chinese Patent Application No. 202410191238.0 filed with the China National Intellectual Property Administration on Feb. 20, 2024, the disclosure of which is incorporated by reference herein in its entirety as part of the present application.

### TECHNICAL FIELD

The present disclosure belongs to the technical field of biomedicine, and in particular relates to the use of a turnip extract in the preparation of a drug for the prevention and treatment of Alzheimer's disease.

### BACKGROUND

Alzheimer's disease (AD) is a neurodegenerative disease characterized by memory and cognitive dysfunction, commonly known as senile dementia. Unlike general aging, AD patients experience rapid memory decline, and even complete loss of logical thinking, and are unable to take care of their lives at an advanced stage. AD is a complex disease whose pathogenesis is unclear. The most typical pathological features of the patient's brain are Aβ amyloid deposition and neurofibrillary tangles formed by hyperphosphorylation of a Tau protein.

Currently, the clinical drugs used in the treatment of AD are mainly chemical drugs, which can only delay the disease progression and result in toxic and side effects, drug resistance and other problems. Traditional Chinese medicine and ethnomedicine are characteristiced by multi-component, multi-target, and multi-pathway, and thus have certain advantages for the treatment of complex diseases such as AD. Therefore, research on anti-AD related drugs based on traditional Chinese medicine and ethnomedicine has become a current hot spot.

Turnip, with the scientific name of Brassicarapa L., is a biannual herb of the genus Brassica in the Cruciferae family, distributed all over China. It is a kind of vegetable that people love to eat. The fleshy root of turnip is the edible part and is also the part recorded in the classics of traditional Chinese medicine and ethnomedicine for medicinal use, possessing relatively high edible and medicinal values. Modern studies have shown that the fleshy root of turnip contains compounds such as flavonoids, phenolic acids and saponins, and has various pharmacological effects including anti-oxidation, anti-hypoxia, anti-tumor, anti-inflammation, hypoglycemic, regulation of immune function, etc. Currently, there are no reports of anti-AD effects of the turnip extract.

### SUMMARY

An objective of the present disclosure is to provide the use of a turnip extract in the preparation of a drug for the prevention and treatment of Alzheimer's disease. The turnip extract of the present disclosure has good activity against Alzheimer's disease and has good application prospects in a drug for the prevention and treatment of Alzheimer's disease.

In order to achieve the objective described above, the present disclosure provides the following technical solutions.

The present disclosure provides the use of a turnip extract in the preparation of a drug for the prevention and treatment of Alzheimer's disease.

A method for preparing the turnip extract includes the steps of:
mixing turnip with an ethanol aqueous solution for an extraction to obtain an extract solution;
subjecting the extract solution to a first concentration to obtain an extractum A;
mixing the extractum A with water to obtain an extractum B;
extracting the extractum B with petroleum ether and collecting a petroleum ether raffinate;
extracting the petroleum ether raffinate with ethyl acetate and collecting an ethyl acetate extract solution;
subjecting the ethyl acetate extract solution to a second concentration to obtain the turnip extract.

In some embodiments, a volume fraction of the ethanol aqueous solution is 60-90%.

In some embodiments, the extraction is conducted for 2-5 times; a solid-liquid ratio for each extraction is 1 g: 5-50 mL.

In some embodiments, the extraction includes an ultrasonic-assisted extraction and leaching performed in sequence; the ultrasonic-assisted extraction is conducted at a power of 60-900 W, a temperature of 30-80° C. and a time of 10-60 min; and the leaching is conducted at 50-80° C. for 1-4 h.

In some embodiments, a volume ratio of the extractum A to water is 1:0.5-10; a volume ratio of the extractum B to petroleum ether is 1:0.5-10; a volume ratio of the petroleum ether raffinate to ethyl acetate is 1:0.5-10.

In some embodiments, a boiling point of the petroleum ether is 60-90° C.

In some embodiments, the first concentration and the second concentration are both vacuum concentration.

In some embodiments, a total flavonoid content of the turnip extract is not less than 40 mg of rutin equivalent/g of the turnip extract.

In some embodiments, a total phenol content of the turnip extract is not less than 20 mg of gallic acid equivalent/g of the turnip extract.

In some embodiments, a dosage form of the drug for the prevention and treatment of Alzheimer's disease includes at least one of a tablet, a pill, a granule, a suspension, an oral liquid and a spray.

The present disclosure provides the use of a turnip extract in the preparation of a drug for the prevention and treatment of Alzheimer's disease. The turnip extract of the present disclosure has good activity against Alzheimer's disease and has good application prospects in the preparation of a drug for Alzheimer's disease. Experimental studies have shown that in an in-vitro PC12 cell model, the turnip extract prepared according to the present disclosure can reduce the level of intracellular reactive oxygen species (ROS) by increasing the antioxidant capacity of the cells, thereby reducing apoptosis; in an in-vivo mouse model, the prepared turnip extract can maintain the integrity of nerve cells and reduce Aβ deposition and p-Tau protein production in the hippocampal region of the brain by reducing the levels of acetylcholinesterase (AChE), β-secretase 1 (BACE1), malondialdehyde (MDA) and inflammatory factors in brain tissues of senile dementia mice, and increasing the levels of antioxidases such as glutathione peroxidase, superoxide dismutase and catalase, thereby exerting an anti-senile dementia effect; and the turnip extract has anti-AD efficacy comparable to the positive control drug donepezil hydrochloride, and can be used as an ideal raw material for anti-AD drugs.

A method for preparing the turnip extract of the present disclosure includes the steps of: mixing turnip with an ethanol aqueous solution for an extraction to obtain an extract solution; subjecting the extract solution to a first concentration to obtain an extractum A; mixing the extractum A with water to obtain an extractum B; extracting the extractum B with petroleum ether and collecting a petroleum ether raffinate; extracting the petroleum ether raffinate with ethyl acetate and collecting an ethyl acetate extract solution; subjecting the ethyl acetate extract solution to a second concentration to obtain the turnip extract. In the present disclosure, the dissolution of active ingredients of flavonoids and phenols can be promoted by an extraction with ethanol aqueous solution, an extraction with petroleum ether and an extraction with ethyl acetate, obtaining a turnip extract rich in active ingredients of flavonoids and phenols, with simple operations. The results of the examples show that the turnip extract prepared using the preparation method provided by the present disclosure has a total flavonoid content of no less than 40 mg of rutin equivalent/g of the turnip extract, and a total phenol content of no less than 20 mg of gallic acid equivalent/g of the turnip extract, and has significant anti-AD activity.

### BRIEF DESCRIPTION OF THE DRAWINGS

In order to illustrate the technical solutions in embodiments of the present disclosure or in the prior art more clearly, a brief introduction to the drawings required for the examples will be provided below. It is obvious that the drawings in the following description only illustrate some of the embodiments of the present disclosure, and those of ordinary skill in the art can also obtain other drawings according to these drawings without involving any inventive effort.

FIGS. 1A-1B show the results for the impact of the turnip extract on the survival rate of PC12 cells damaged by H₂O₂;

FIG. 2 shows the results for the impact of the turnip extract on flow cytometry testing of ROS in PC12 cells damaged by H₂O₂;

FIG. 3 shows the results for the impact of the turnip extract on the fluorescence intensity of ROS in PC12 cells damaged by H₂O₂;

FIGS. 4A-4D show the results for the impact of the turnip extract on PC12 cells damaged by H₂O₂;

FIG. 5 shows the results for the impact of different concentrations of the turnip extract on the changes in the body weight of the mice;

FIG. 6 shows the results for the impact of different concentrations of the turnip extract on the latency of the mice during the hidden platform period;

FIGS. 7A-7C show the results for the impact of different concentrations of the turnip extract on the movement trajectory, the number of times of crossing the original platform, and the percentage of the retention time in the quadrant of the original platform of the mice during the platform-free period;

FIGS. 8A-8B show the results for the impact of different concentrations of the turnip extract on AChE and BACE1 activity in the brains of mice;

FIGS. 9A-9D show the results for the impact of different concentrations of the turnip extract on superoxide dismutase activity, glutathione peroxidase activity, catalase activity and MDA level in the brains of mice;

FIGS. 10A-10B show the results for the impact of different concentrations of the turnip extract on inflammatory factors TNF-α and IL-6 in the brains of mice;

FIG. 11 shows the results for HE staining of brain tissue sections of mice in all groups;

FIG. 12 shows the results for Aβ immunohistochemical staining of brain tissue sections of the mice in all groups;

FIG. 13 shows the results for p-Tau protein immunohistochemical staining of brain tissue sections of the mice in all groups.

### DETAILED DESCRIPTION OF THE EMBODIMENTS

The present disclosure provides the use of a turnip extract in the preparation of a drug for the prevention and treatment of Alzheimer's disease.

A method for preparing the turnip extract includes the steps of:
mixing turnip with an ethanol aqueous solution for an extraction to obtain an extract solution;
subjecting the extract solution to a first concentration to obtain an extractum A;
mixing the extractum A with water to obtain an extractum B;
extracting the extractum B with petroleum ether and collecting a petroleum ether raffinate;
extracting the petroleum ether raffinate with ethyl acetate and collecting an ethyl acetate extract solution;
subjecting the ethyl acetate extract solution to a second concentration to obtain the turnip extract.

In the present disclosure, unless otherwise specified, the raw materials used are commercially available products well known to those skilled in the art or are prepared by methods well known to those skilled in the art.

In the present disclosure, turnip is mixed with an ethanol aqueous solution for extraction to obtain an extract solution. In the present disclosure, in some embodiments, before performing the extraction, turnip is pulverized and dried to obtain a turnip powder. In some embodiments, a particle size of the turnip powder of the present disclosure is preferably not lower than 40 mesh. In examples of the present disclosure, the following steps are in particular included: Fresh turnip is washed with clean water, air dried, cut into 3-5 mm slices, and then dried in the shade. The slices are crushed by a crusher and passed through a 40 mesh screen to obtain a turnip powder for later use. In the present disclosure, after obtaining the turnip powder, the turnip powder is mixed with an ethanol aqueous solution for extraction. In the present disclosure, in some embodiments, a volume fraction of the ethanol aqueous solution is 60-90%, more preferably 80%. In the present disclosure, in some embodiments, the extraction is conducted for 2-5 times, more preferably 3 times; in some embodiments, a solid-liquid ratio for each extraction is 1 g:5-50 mL, more preferably 1 g:10-25 mL. The extraction of the present disclosure preferably includes an ultrasonic-assisted extraction and leaching performed in sequence. In some embodiments, the ultrasonic-assisted extraction of the present disclosure is conducted at a powder of 60-900 W, more preferably 300-500 W; a temperature of 30-80° C., more preferably 50-60° C.; a time of 10-60 min, more preferably 20-30 min. In some embodiments, the leaching of the present disclosure is conducted at 50-80° C., more preferably 50-60° C., for 1-4 h, more preferably 2-3 h. After each extraction is completed, the material liquid obtained from the extraction is subjected to a solid-liquid separation to obtain a supernatant and a filter residue. The filter residue is subjected to the next extraction. The supernatant from each extraction is combined to obtain an extract solution. In some embodiments, a method for the solid-liquid separation of the present disclosure is filtration.

In the present disclosure, after obtaining the extract solution, the extract solution is subjected to a first concentration to obtain an extractum A. In the present disclosure, in some embodiments, the first concentration is vacuum concentration; the vacuum concentration is conducted at 30-80° C., preferably 50-60° C. In the present disclosure, in some embodiments, the extract solution is vacuum concentrated until no alcohol smell exists, and the extractum A is obtained. In the present disclosure, in some embodiments, an instrument for performing the first concentration is a vacuum rotary evaporator.

In the present disclosure, after obtaining the extractum A, the extractum A is mixed with water to obtain an extractum B. In the present disclosure, in some embodiments, a volume ratio of the extractum A to water is 1:0.5-10, preferably 1:1-5.

In the present disclosure, after obtaining the extractum B, the extractum B is extracted with petroleum ether, and a petroleum ether raffinate is collected. In the present disclosure, in some embodiments, a boiling point of the petroleum ether is 60-90° C., preferably 70-80° C. In the present disclosure, in some embodiments, a volume ratio of the extractum B to petroleum ether is 1:0.5-10, preferably 1:1-5. In the present disclosure, the extraction is performed until a petroleum ether extract solution becomes colorless, then the petroleum ether extract solution is discarded, and a petroleum ether raffinate is obtained.

In the present disclosure, after obtaining the petroleum ether raffinate, the petroleum ether raffinate is extracted with ethyl acetate, and an ethyl acetate extract solution is collected. In the present disclosure, in some embodiments, a volume ratio of the petroleum ether raffinate to ethyl acetate is 1:0.5-10, preferably 1:1-5. In the present disclosure, the extraction is performed until an ethyl acetate extract solution becomes colorless, then an ethyl acetate raffinate is discarded, and a resulting ethyl acetate extract solution is obtained.

In the present disclosure, after obtaining the ethyl acetate extract solution, the ethyl acetate extract solution is subjected to a second concentration to obtain the turnip extract. In the present disclosure, in some embodiments, the second concentration is vacuum concentration, the vacuum concentration is conducted at 40-65° C., preferably 50-60° C. In the present disclosure, in some embodiments, after the second concentration, the obtained concentrated material is subjected to drying. In the present disclosure, in some embodiments, the drying is vacuum freeze-drying (lyophilization).

In the present disclosure, in some embodiments, a total flavonoid content of the turnip extract is not less than 40 mg of rutin equivalent/g of the turnip extract, and a total phenol content is not less than 20 mg of gallic acid equivalent/g of the turnip extract.

In the present disclosure, a dosage form of the drug for the prevention and treatment of Alzheimer's disease includes at least one of a tablet, a pill, a granule, a suspension, an oral liquid and a spray.

The technical solutions in the present disclosure will be described clearly and completely below with reference to the examples of the present disclosure. Apparently, the described examples are only some, but not all, examples of the present disclosure. On the basis of the examples of the present disclosure, all the other examples that would have been obtained by those of ordinary skill in the art without involving any inventive effort shall fall within the scope of protection of the present disclosure.

### EXAMPLE 1

(1) fresh turnip was cleaned with water, air dried, cut into 3-5 mm slices, and then dried in the shade. The slices were crushed using a crusher and passed through a 40-mesh sieve to obtain a turnip for later use.

1 kg of turnip powder was accurately weighed, and an ethanol aqueous solution with a volume fraction of 80% was added (at a solid-liquid ratio of 1:10 g/mL). Firstly, ultrasonic-assisted extraction was performed for 20 min (at a temperature of 50° C. and a power of 300 W). Then, the ultrasonic device was turned off, and leaching was performed in a water bath at 60° C. for 2 h to obtain a leach solution.

The leach solution was filtered with gauze to obtain a supernatant and a filter residue. The filter residue was repeatedly extracted twice under the conditions described above. The supernatants obtained after three leachings were combined and concentrated using a vacuum rotary evaporator at 50° C. until no alcohol smell exists, and an extractum A was obtained.
(2) An equal volume of distilled water was added to the extractum A. After mixing uniformly, an extractum B with good fluidity was obtained.
(3) The extractum B was extracted with an equal volume of petroleum ether (bp, 60-90° C.) until a petroleum ether extract solution became colorless, then the petroleum ether extract solution was discarded, and a petroleum ether raffinate was obtained.
(4) The petroleum ether raffinate was extracted with an equal volume of ethyl acetate until an ethyl acetate extract solution became colorless, then an ethyl acetate raffinate was discarded, and a resulting ethyl acetate extract solution was collected.
(5) The ethyl acetate extract solution was vacuum concentrated at 50° C. The concentrated material was lyophilized to obtain a turnip extract.

The total flavonoid content of the turnip extract was determined by the aluminum trichloride colorimetric method, which was 55 mg of rutin equivalent/g of the turnip extract. The total phenol content of the turnip extract was determined by the Folin-Ciocalteu method, which was 25 mg of gallic acid equivalent/g of the turnip extract.

### TEST EXAMPLE 1

### Evaluation of In-Vitro Anti-AD Activity of the Turnip Extract Prepared in Example 1

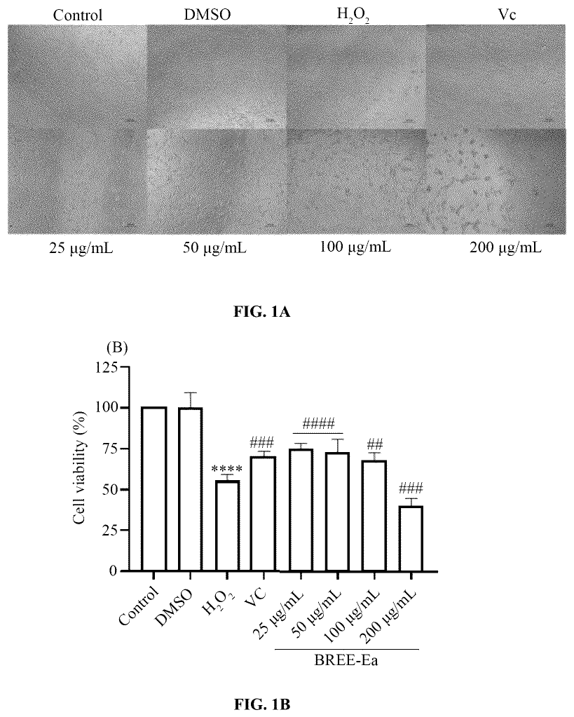

Rat adrenal pheochromocytoma cells (PC12 cells) were treated with different concentrations of the turnip extract (25 μg/mL, 50 μg/mL, 100 μg/mL and 200 μg/mL). After 24 h, the supernatant was removed. 100 μL of H₂O₂ at a concentration of 200 μmol/L was added to each well, and the cells were treated for 4 h. Then the treated PC12 cells were tested as follows:
(1) The morphology of the treated PC12 cells was observed under a microscope. The results are shown in FIG. 1A.

The experimental results showed that the cells in the H₂O₂-induced damage model group became round and reduced in number, whereas the turnip extract could reduce the degree of cell damage.
(2) The viability of the treated PC12 cells was detected using the 3-(4,5-dimethylthiazole-2)-2,5-diphenyltetrazolium bromide method (MTT method). The results are shown in FIG. 1B (significant differences in the figure are denoted as follows: ****p<0.0001 compared with the blank group; ##p<0.01, ###p<0.001, ####p<0.0001 compared with the H₂O₂ group.

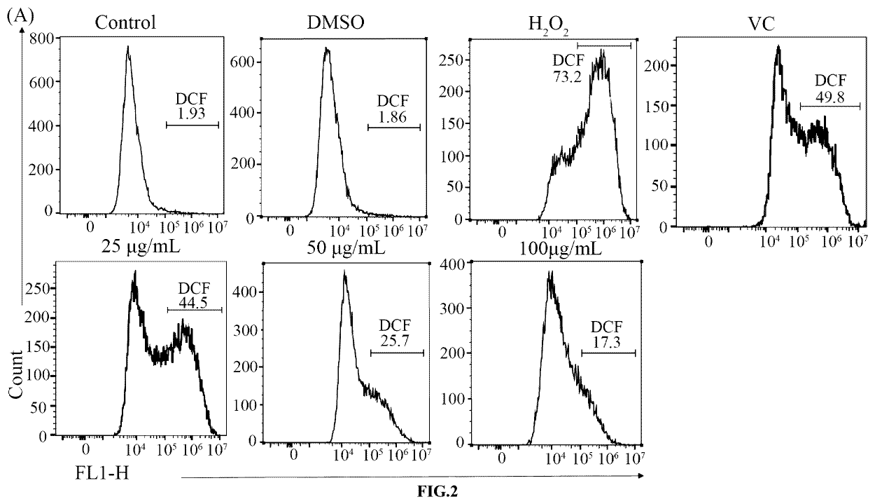

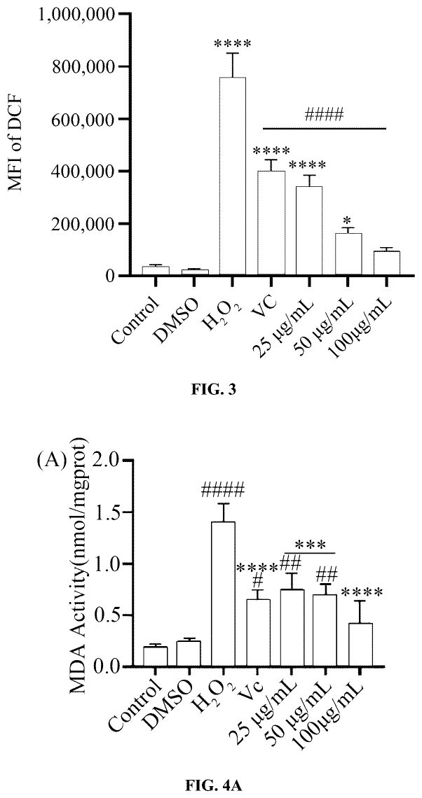

The experimental results showed that the turnip extracts in the medium-to low-dose groups (25 μg/mL, 50 μg/mL, and 100 μg/mL) all could significantly increase the survival rate of PC12 cells, with the low-dose group (25 μg/mL) performing the best.
(3) The detection results of oxidative damage indicators of the treated PC12 cells, such as MDA, lactate dehydrogenase, glutathione peroxidase, superoxide dismutase, and reactive oxygen species (ROS) produced in the cells, are shown in FIGS. 2-3.

FIG. 2 shows the results for the impact of the turnip extract on flow cytometry testing of ROS in PC12 cells damaged by H₂O₂.

FIG. 3 shows the results for the impact of the turnip extract on the fluorescence intensity of ROS in PC12 cells damaged by H₂O₂.

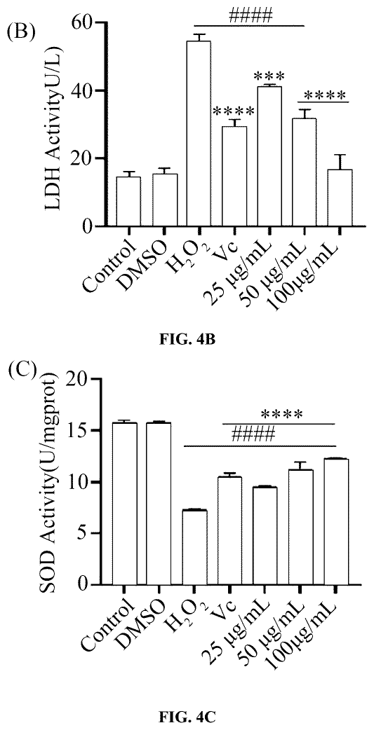

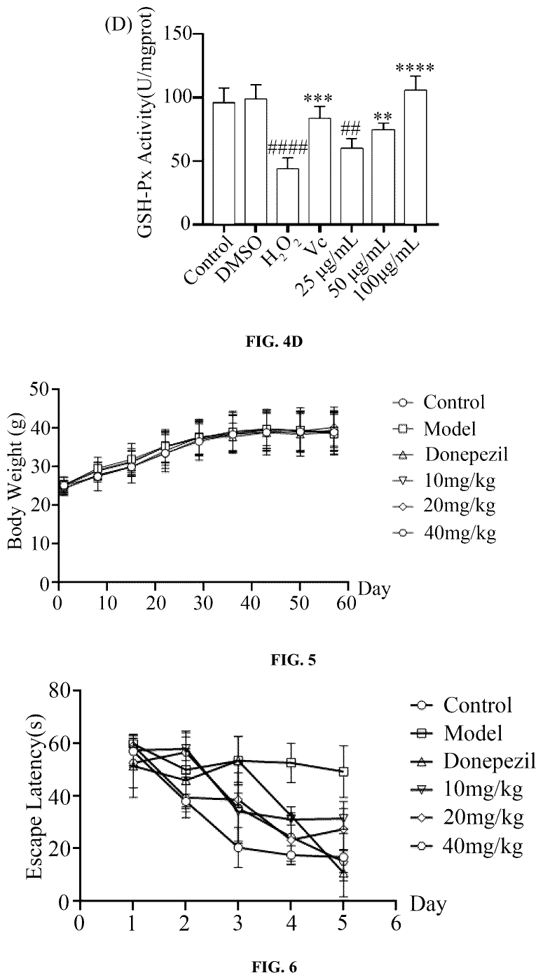

FIGS. 4A-4D show the results for the impact of the turnip extract on PC12 cells damaged by H₂O₂, in which FIG. 4A shows the results for the impact of the turnip extract on the MDA content of PC12 cells damaged by H₂O₂, FIG. 4B shows the results for the impact of the turnip extract on the lactate dehydrogenase activity of PC12 cells damaged by H₂O₂, FIG. 4C show the results for the impact of the turnip extract on the superoxide dismutase level of PC12 cells damaged by H₂O₂, and FIG. 4D show the results for the impact of the turnip extract on glutathione peroxidase of PC12 cells damaged by H₂O₂.

The experimental results showed that the turnip extract could increase the levels of glutathione peroxidase and superoxide dismutase and reduce the levels of MDA and lactate dehydrogenase in a concentration-dependent manner, thereby reducing intracellular ROS, and effectively reducing apoptosis.

### TEST EXAMPLE 2

### Evaluation of In-Vivo Anti-AD Activity of the Turnip Extract

To evaluate the in-vivo anti-AD effect of the turnip extract, a model of senile dementia was established by intraperitoneal injection of 0.2 mL of D-galactose (120 mg/kg) and NaNO₂ (90 mg/kg) for a total of 60 days. The changes in the body weight of the mice were recorded during the experiment. 30 days after modeling, the mice were randomly divided into a model group, a positive control group, a low-dose group (25 μg/mL), a medium-dose group (50 μg/mL) and a high-dose group (100 μg/mL). The mice in the dose groups were subjected to intragastric administration of 0.2 mL of the corresponding test drugs, respectively, and the mice in the blank control group and the model group were subjected to intragastric administration of 0.2 mL of normal saline for 30 days. Half an hour after the last dose, a behavioral test was started. The day after the behavioral experiment, the mice were sacrificed by cervical dislocation, and the brains were cut along the midline. Half of the brain tissue was prepared into 10% brain tissue homogenate with cold normal saline, and the other half was fixed in a paraformaldehyde fixative with a mass fraction of 4% for the preparation of sections.

### (1) Results for the Impact of Different Concentrations of the Turnip Extract on the Mice

FIG. 5 shows the results for the impact of different concentrations of the turnip extract on the changes in the body weight of the mice.

FIG. 6 shows the results for the impact of different concentrations of the turnip extract on the latency of the mice during the hidden platform period.

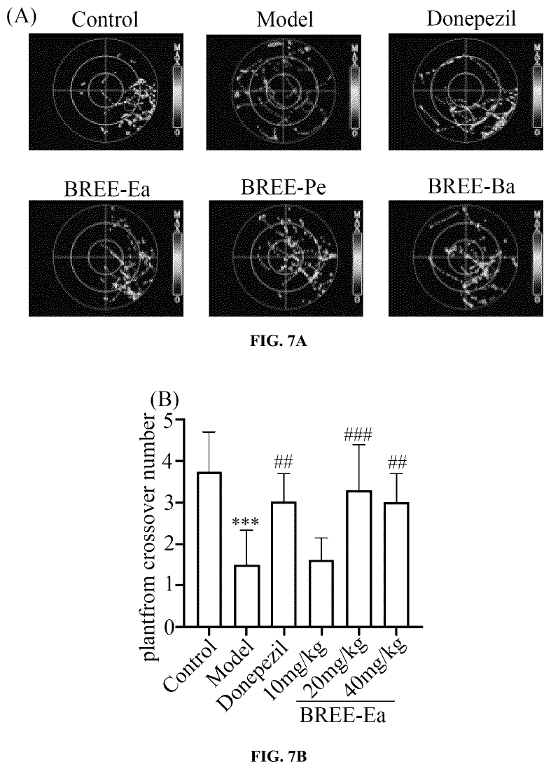

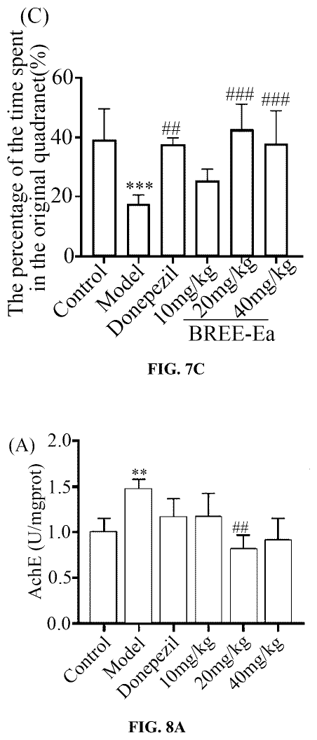

FIGS. 7A-7C show the impact of different concentrations of the turnip extract on the movement trajectory, the number of times of crossing the original platform, and the percentage of the retention time in the quadrant of the original platform of the mice during the platform-free period, in which FIG. 7A shows the impact of different concentrations of the turnip extract on the movement trajectory of the mice during the platform-free period; FIG. 7B shows the impact of different concentrations of the turnip extract on the number of times of crossing the original platform of the mice during the platform-free period, and FIG. 7C shows the impact of different concentrations of the turnip extract on the percentage of the retention time in the quadrant of the original platform of the mice during the platform-free period.

The results showed that the administration of the turnip extract had no significant impact on the changes in the body weight of the mice (FIG. 5); compared with the mice in the senile dementia model group, the latency of the mice in the middle dose group during the hidden platform period was decreased (FIG. 6), and the memory and cognitive capacities during the platform-free period were significantly improved (FIGS. 7A-7C).

### (2) Detection Results for Related Physicochemical Indicators in the Brains of Mice

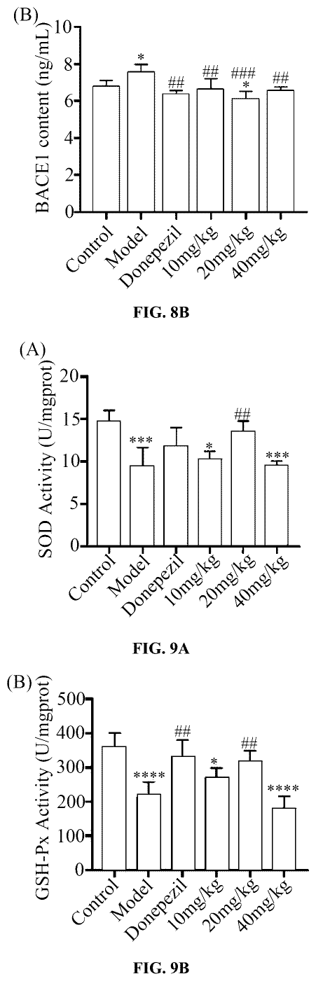

FIGS. 8A-8B show the results for the impact of different concentrations of the turnip extract on AChE and BACE1 activity in the brains of mice, in which FIG. 8A shows the impact of different concentrations of the turnip extract on AChE activity in the brains of mice, and FIG. 8B shows the impact of different concentrations of the turnip extract on BACE1 activity in the brains of mice.

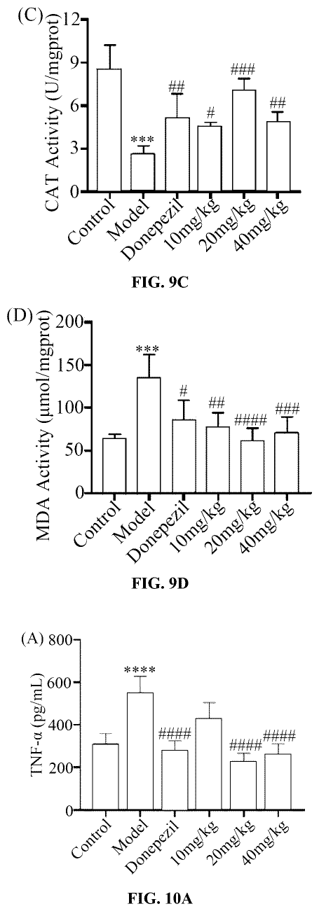

FIGS. 9A-9D show the results for the impact of different concentrations of the turnip extract on superoxide dismutase activity, glutathione peroxidase activity, catalase activity and MDA level in the brains of mice, in which FIG. 9A shows the results for the impact of different concentrations of the turnip extract on superoxide dismutase activity in the brains of mice, FIG. 9B shows the results for the impact of different concentrations of the turnip extract on glutathione peroxidase activity in the brains of mice, FIG. 9C shows the results for the impact of different concentrations of the turnip extract on catalase activity in the brains of mice, and FIG. 9D shows the results for the impact of different concentrations of the turnip extract on MDA level in the brains of mice.

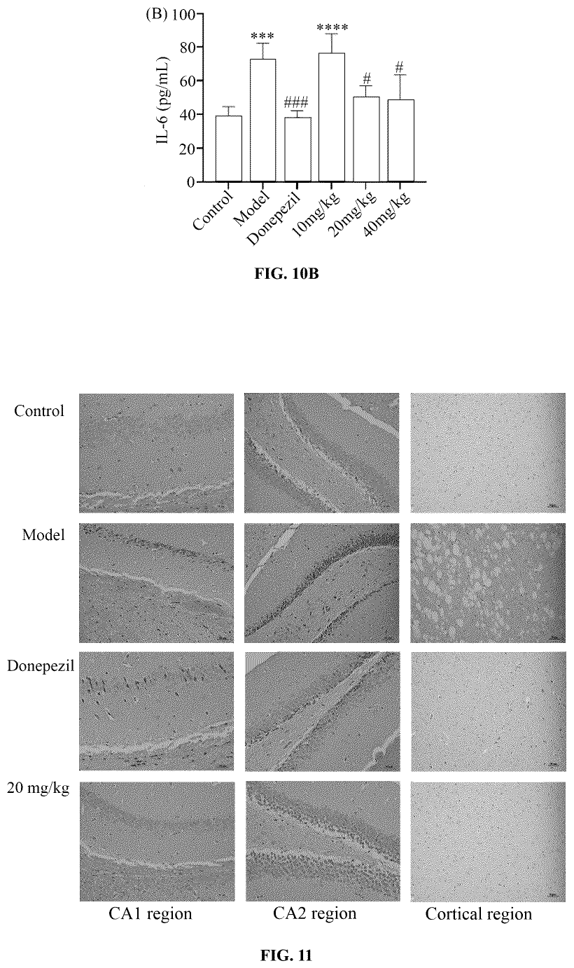

FIGS. 10A-10B shows the results for the impact of different concentrations of the turnip extract on inflammatory factors TNF-α and IL-6 in the brains of mice, in which FIG. 10A shows the impact of different concentrations of the turnip extract on the inflammatory factor TNF-α in the brains of mice, and FIG. 10B shows the impact of different concentrations of the turnip extract on the inflammatory factor IL-6 in the brains of mice.

The results showed that compared with the mice in the senile dementia model group, the turnip extract could reduce the levels of AChE and BACE1 in the brain tissues of the mice (FIGS. 8A-8B), increase the activity of antioxidases such as glutathione peroxidase, superoxide dismutase and catalase (FIG. 9B, 9A and 9C), and reduce the content of a lipid peroxide MDA (FIG. 9D) and the inflammation level (FIGS. 10A-10B), thereby increasing the cognitive ability of the mice and exerting anti-AD activity. These results showed that the turnip extract at 20 mg/kg could have the greatest effect on protecting the memory capacity of senile dementia mice and enhancing the cognitive function of senile dementia mice.

### (3) Results for Staining of Brain Tissue Sections of Mice

FIG. 11 shows the results for hematoxylin-eosin (HE) staining of brain tissue sections of the mice in all groups.

The results for HE staining showed that in the senile dementia mice, the number of cells in the hippocampal region was reduced, the cells were shrunken in morphology, and there were vacuolation phenomena in the cortical region, which could be significantly improved by treatment with the medium-dose turnip extract, and the hippocampal region and the cortical region of the mice approached the mice in normal group.

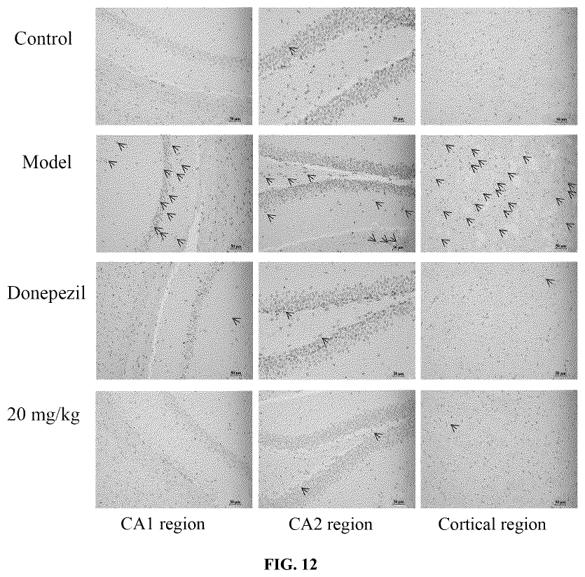

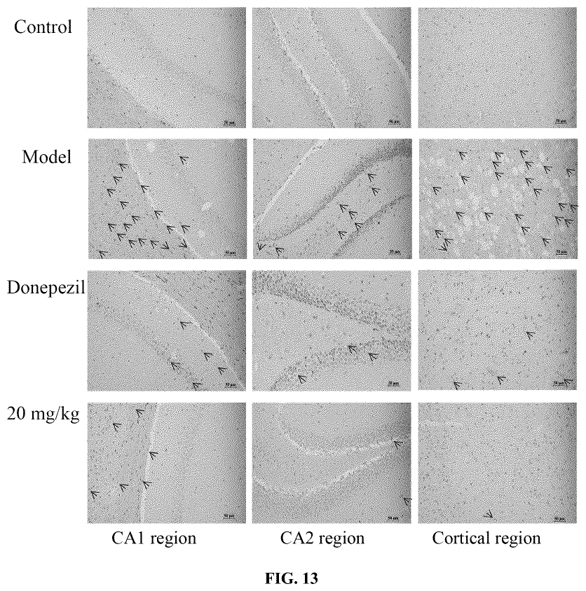

FIG. 12 shows the results for Aβ immunohistochemical staining of brain tissue sections of the mice in all groups; FIG. 13 shows the results for p-Tau protein immunohistochemical staining of brain tissue sections of the mice in all groups.

The results for immunohistochemical staining showed that the Aβ protein and the phosphorylated Tau protein were deposited more in the hippocampal region and the cortical region of senile dementia mice and brown spots were significant. After treatment with the medium-dose turnip extract, the deposition of the Aβ protein and the phosphorylated Tau protein in the hippocampal region and the cortical region could be improved and brown spots were reduced (FIG. 12 and FIG. 13).

In the present disclosure, the anti-AD activity of the turnip extract was evaluated using different methods, including in-vitro and in-vivo experiments for detailed verifications, with the mechanism of action demonstrated, and it was found that the turnip extract had a good anti-AD effect.

Although the examples described above have provided a detailed description of the present disclosure, they are only a part of, rather than all of the examples of the present disclosure. All other examples that can be obtained according to the examples of the present disclosure without involving any inventive effort shall fall within the scope of protection of the present disclosure.

What is claimed is:

1. A method for preventing and treating Alzheimer's disease, the method comprising: administrating turnip extract to a subject in need;
wherein the turnip extract is prepared by:
mixing turnip with an ethanol aqueous solution for an extraction to obtain an extract solution;
subjecting the extract solution to a first concentration to obtain an extractum A;
mixing the extractum A with water to obtain an extractum B;
extracting the extractum B with petroleum ether and collecting a petroleum ether raffinate;
extracting the petroleum ether raffinate with ethyl acetate and collecting an ethyl acetate extract solution; and
subjecting the ethyl acetate extract solution to a second concentration to obtain the turnip extract.

2. The method according to claim 1, wherein a volume fraction of the ethanol aqueous solution is 60-90%.

3. The method according to claim 1, wherein the extraction is conducted for 2-5 times; and a solid-liquid ratio for each extraction is 1 g: 5-50 mL.

4. The method according to claim 3, wherein the extraction comprises an ultrasonic-assisted extraction and leaching performed in sequence; the ultrasonic-assisted extraction is conducted at a power of 60-900 W, a temperature of 30-80° C. and a time of 10-60 min; and the leaching is conducted at 50-80° C. for 1-4 h.

5. The method according to claim 1, wherein a volume ratio of the extractum A to water is 1:0.5-10; a volume ratio of the extractum B to petroleum ether is 1:0.5-10; a volume ratio of the petroleum ether raffinate to ethyl acetate is 1:0.5-10.

6. The method according to claim 1, wherein a boiling point of the petroleum ether is 60-90° C.

7. The method according to claim 1, wherein the first concentration and the second concentration are vacuum concentration.

8. The method according to claim 1, wherein a total flavonoid content of the turnip extract is not less than 40 mg of rutin equivalent/g of the turnip extract.

9. The method according to claim 1, wherein a total phenol content of the turnip extract is not less than 20 mg of gallic acid equivalent/g of the turnip extract.

10. The method according to claim 1, wherein the turnip extract is provided in a drug with a dosage form comprising at least one of a tablet, a pill, a granule, a suspension, an oral liquid and a spray.

11. The method of claim 3, wherein a volume fraction of the ethanol aqueous solution is 60-90%.

12. The method of claim 6, wherein a volume ratio of the extractum A to water is 1:0.5-10; a volume ratio of the extractum B to petroleum ether is 1:0.5-10; a volume ratio of the petroleum ether raffinate to ethyl acetate is 1:0.5-10.
